(19) United States
(12) Patent Application Publication
(10) Pub. No.: US 2003/0118525 A1
(43) Pub. Date: Jun. 26, 2003

(54) Treatment of uv induced immunosuppression

(76) Inventor: Geoffrey Walter Grigg, Lane Cova (AU)

Correspondence Address: MCDERMOTT WILL & EMERY, 600 13TH STREET, N.W., WASHINGTON, DC, 20005-3096, US

(21) Appl. No.: 10/181,524

(22) PCT Filed: Jan. 17, 2001
(86) PCT No.: PCT/AU01/00039

(30) Foreign Application Priority Data
Jan. 19, 2000 (AU) PQ 5150

(51) Int. Cl. A61K 31/195; A61K 7/42

(52) U.S. Cl. 424/59; 514/563

(57) ABSTRACT

The present invention relates to methods for the treatment or prevention of skin damage and/or UV-induced immuno-suppression. In particular, the invention relates to methods for the treatment or prevention of UV-induced immunosuppression, skin erythenma and skin collagen damage which involve the administration of carnosine, acycarnosine or related compounds.

### FIELD OF THE INVENTION

The present invention relates to methods for the treatment or prevention of skin damage and/or UV-induced immunosuppression. In particular, the invention relates to treatment or prevention of UV-induced immunosuppression, skin erythema and skin collagen damage.

### BACKGROUND OF THE INVENTION

The exposure of skin to solar radiation induces a number of changes of both temporary and permanent nature to its structure. The active agent in solar radiation is the ultraviolet region of its spectrum. The changes induced from damage to the DNA of the skin cells to proteins and lipids in these cells or matrix components such as collagen or elastin between the cells. In turn these cellular insults induce an inflammatory tissue reaction and various secondary consequences ranging from mutation in cells, protein cross linking in the matrix of the epidermis and the dermis, loss of immuno-competence involving loss of responsiveness of the skin's dendritic cells, tissue oedema and in time a loss of elasticity in the skin, greater frequency of skin cancers, pigmented senile plaques, stimulation of pigment production by melanocytes in the epidermis or coarsening of the surface of the skin. In general, exposure of skin to solar radiation accelerates premature aging as judged by both the appearance of the skin and loss of physiological function.

These undesirable consequences can be modulated by preventing the damaging radiation from reaching its target by screening the skin in some way such as covering the skin with clothing or a film of a substance which through solar radiation does not penetrate.

Whilst the role of the UV-B portion (290 nm to 320 nm) of the spectrum of solar radiation to the induction of erythema, of basal cell carcinoma and of immunosuppression of the skin is established, the significance of UV-A (320 nm to 400 nm) rays in skin pathogenesis and skin aging is less clear. The fact that the longer wavelength UV-A radiation penetrates far lower into the skin than does UV-B makes it a potentially important factor as a skin damaging agent.

Recent data from a long term trial of UV-B sunscreens demonstrate effective protection against the production of one type of sunlight induced skill cancer, squamous cell carcinoma, but not against basal cell carcinoma or melanoma. One inference from this large scale trial is that some other component of solar radiation is responsible for the latter malignancies. Presumably, that component is UV-A.

UV-A radiation may also lead to damage to the elastic and collagenic fibers of connective tissue. Furthermore, it causes numerous phototoxic and photoallergic reactions. The damaging effects of UV-B radiation can also be intensified by UV-A radiation.

It is also known that UV-B radiation can impair a variety of immune responses in humans and laboratory animals both locally, within UV-irradiated skin, and systemically, at distance sites (1). Exposure of mice to UV-B radiation interferes with the rejection of UV-induced skin cancers and the induction of delayed and contact hypersensitivity (DHS, CHS) responses initiated in unirradiated sites; these forms of immune suppression are associated with the induction of antigen-specific suppressor T lymphocytes (2). How UV-B radiation exerts its systemic, immunosuppressive effects is a question of considerable interest, both for understanding the regulatory pathways governing these immune responses and for assessing the potential effects of UV-B radiation on human health. The CHS response is particularly important in this regard because this T-lymphocyte-mediated immune reaction is responsible for protection against many infectious diseases.

Current experimental evidence implicates soluble substances derived from UV-irradiated keratinocytes as the probable mediators of UV-induced systemic suppression of DHS and CHS responses (3-5). However, the initial photobiological reaction responsible for triggering the cascade of events leading to activation of the suppressor pathway of the immune response remains controversial.

The dipeptide carnosine (βalanyl-L-histidine) is non-toxic and tasteless, and it is known that experimental animals can tolerate high levels in their diet. For example, mice with 5% of carnosine in their food appear to be unaffected, and the subsequent distribution of carnosine in various tissues of these animals has been determined. Exogenous carnosine levels are highest in liver, spleen and kidney, and at lower levels in brain, muscle and serum. It should be noted that in normal mice which have not been fed carnosine have high endogenous carnosine levels in muscle. Mice have also been treated with 3% carnosine in drinking water with no outward effect. Several studies over many species of mammals including man indicate that the level of carnosine in skeletal muscle is correlated with species lifespan.

Carnosine is known to have a range of activities including preventing non-enzymic glycation of proteins; antioxidative properties; slowing down the aging process in human fibroblasts; chelating transition metal ions; protecting the vascular system against ischemia; and acting as an effective buffering agent. It is also known to be useful in preventing cataract formation in diabetic animals. Although carnosine has been shown to protect bacteriophages from the effects of ionising radiation and appears to efficiently scavenge hydroxy radicals, it is not as efficient at quenching certain other radicals.

In previous studies (6), lotions containing 0.1% to 1.0% w/v carnosine applied topically to the skin of hairless mice (100 μl lotion daily) before irraditation, were demonstrated to protect the immune suppressive effects of simulated solar radiation. The lotions were applied to the dorsal skin prior to daily exposure to the simulated solar radiation. In these experiments the lotion application was continued daily for 12 days (the total time for induction of contact hypersensitivity to 2,4-dinitrofluorobenzene (DNFB)).

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this application.

### DISCLOSURE OF THE INVENTION

The present inventors have now found that carnosine and acyl carnosines are surprisingly effective in blocking the erythema and attendant oedema induced by exposure of the skin to solar radiation. Application of the active agent either before or after the irradiation exposure is effective.

Accordingly, in a first aspect the present invention provides a method for preventing erythema or inflammatory cascade effects caused by solar radiation in a subject which method comprises administering to the subject at least one compound selected from the group consisting of carnosine, a compound related to carnosine, an acylcarnosine and a compound related to an acylcarnosine.

As will be appreciated by those skilled in the art, inflammatory cascade effects caused by solar radiation include effects such as reddening of the skin, tissue oedema and blistering of the skin.

In a preferred embodiment of the first aspect, the compound is administered after the subject has been exposed to solar radiation.

The present inventors have also found that acylcarnosines are surprisingly more effective in blocking the immunosuppressive effects of solar radiation than is carnosine. Unlike carnosine, acylderivatives are effective in preventing immunosuppression even when applied after exposure to solar radiation.

Results obtained by the present inventors indicate that acylcarnosines are surprising effective in (a) slowing aging and/or rejuvenating aging cells; (b) blocking the immunosuppressive effects of solar radiation; and (c) reducing the crosslinking between collagen molecules induced by UV radiation. Acyl carnosines exert these effects at low concentrations where the parental compound (carnosine) is ineffective.

Accordingly, in a second aspect the present invention provides a method for the treatment or prevention of UV induced suppression of an immune response in a subject, which method comprises administering to the subject a therapeutically or prophylactically effective amount of at least one acylcarnosine or a compound related to an acylcarnosine.

In a third aspect the present invention provides a method for the treatment or prevention of LW induced suppression of a T-cell mediated immune response in a subject, which method comprises administering to the subject a therapeutically or prophylactically effective amount of at least one acylcarnosine or a compound related to an acylcarnosine.

In a fourth aspect the present invention provides a method for preventing sunburn effects of UV radiation in a subject which method comprises administering to the subject at least one acylcarnosine or a compound related to an acylcarnosine.

In a fifth aspect the present invention provides a method for reducing cross-linking of collagen molecules in skin and/or damage to skin cell DNA which method comprises administering to the skin at least one acylcarnosine or a compound related to an acylcarnosine.

In the context of the fifth aspect, the skin cells may be in an in vitro or an in vivo environment.

In one embodiment of the fifth aspect, the cross-linking is induced by UV radiation.

Illustrative examples of compounds related to carnosine which may be used in the present invention include anserine, ophidine, homocarnosine, homoanserine, D-carnosine and carcinine.

Illustrative examples of acylcarnosines which may be used in the present invention include N-acetylcarnosine, N-butyl-carnosine, propionyl-carnosine and hexyl-carnosine.

Illustrative examples of compounds related to acylcarnosines which maybe used in the present invention include esters of acylcarnosines.

In a preferred embodiment the methods of the present invention further comprises administering topically to the subject a sunscreen agent.

In a further preferred embodiment of the present invention, the carnosine, acylcarnosine or related compound may be administered in combination with one or more other antioxidants such as vitamin E, lipoic acid, cysteine and cysteine derivatives such as N-acetylcysteine, folic acid, phytic acid, citric acid, lactic acid, zinc oxide, ubiquinone and the like or other agents, such as urocanic acid or derivatives or analogues thereof, which are known to protect against UV damage to the skin.

In the context of the present invention, administering the carnosine, acylcarnosine or related compound can be effected or performed using any of the various methods and delivery systems known to those skilled in the art. The administration can be performed, for example, intravenously, orally, via implant, transmucosally, transdermally, topically, intramuscularly, subcutaneously or extracorporeally. In addition, the instant pharmaceutical compositions ideally contain one or more routinely used pharmaceutically acceptable carriers. Such carriers are well known to those skilled in the art. The following delivery systems, which employ a number of routinely used carriers, are only representative of the many embodiments envisioned for administering the instant composition.

Transdermal delivery systems include patches, gels, tapes and creams, and can contain excipients such as solubilizers, permeation enhancers (e.g., fatty acids, fatty acid esters, fatty alcohols and amino acids), hydrophilic polymers (e.g., polycarbophil and polyvinylpyrolidone), and adhesives and tackifiers (e.g., polyisobutylenes, silicone-based adhesives, acrylates and polybutene).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and, derivatives, and hydrophilic polymers such as hydroxypropylmethylcellulose and hyaluronic acid).

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

Topical delivery systems include, for example, gels and solutions, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In the preferred embodiment, the pharmaceutically acceptable carrier is a liposome or a biodegradable polymer. Examples of liposomes which can be used in this invention include the following: (1) CellFectin, 1:1.5 (M/M) liposome formulation of the cationic lipid N,Nl,NII,NIII-tetramethyl-NNI,NII,NIII-tetrapalmitylspermine and dioleoyl phosphatidyl-ethanolamine (DOPE)(GIBCO BRL); (2) Cytofectin GSV, 2:1 (M/M) liposome formulation of a cationic lipid and DOPE (Glen Research); (3) DOTAP (N-[1-(2,3-dioleoyloxy)-N,N,N-trimethyl-ammoniummethylsulfate) (Boehringer Manheim); and (4) Lipofectamine, 3:1 (M/M) liposome formulation of the polycationic lipid DOSPA and the neutral lipid DOPE (GIBCO BRL).

Cosmetic and dermatological formulations which are in the form of a sunscreen agent are particularly preferred. These preferably additionally comprise at least one UV-A filter and/or at least one further UV-B filter and/or at least one inorganic pigment.

Cosmetic and dermatological formulations according to the invention for protection of the skin against UV rays can be in various forms, such as are usually employed, for example, for this type of formulation. They can thus be, for example, a solution, an emulsion of the water-in-oil type (W/O) or of the oil-in-water type (O/W), or a multiple emulsion, for example of the water-in-oil-in-water (W/O/W) type, a gel, a solid stick or else an aerosol.

The cosmetic and dermatological formulations according to the invention can comprise cosmetic auxiliaries such as are usually used in such formulations, for example preservatives, bactericides, perfumes, substances for preventing foaming, dyes, pigments which have a coloring action, thickeners, surface-active substances, emulsifiers, softening humidifying and/or humectant substances, fats, oils, waxes or other customary constituents of a cosmetic or dermatological formulation, such as alcohols, polyols, polymers, foam stabilizers, electrolytes, organic solvents or silicone derivatives.

If the cosmetic or dermatological formulations is a solution or lotion, solvents which can be used are:

water or aqueous solutions;

oils, such as triglycerides of capric or of caprylic acid, but preferably castor oil;

fats, waxes and other naturally occurring and synthetic fat substances, preferably esters of fatty acids with alcohols of low C number, for example with isopropanol, propylene glycol or glycerol, or esters of fatty alcohols with alkanoic acids of low C number or with fatty acids;

alcohols, diols or polyols of low C number and ethers thereof, preferably ethanol, isopropanol, propylene glycol, glycerol, ethylene glycol, ethylene glycol monoethyl or monobutyl or monobutyl ether, diethylene glycol monomethyl or monoethyl ether and analogous products.

Mixtures of the abovementioned solvents are used in particular. Water can be a further constituent of alcoholic solvents.

Emulsions according to the invention, for example in the form of a sunscreen cream or a sunscreen milk, are preferred and comprise, for example, the fats, oils, waxes and other fat substances mentioned, as well as water and an emulsifier such as is usually used for such a type of formulation.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The present invention will now be described more fully with reference to the following non-limiting examples and figures.

### BRIEF DESCRIPTION OF THE FIGURES

FIG. 1 shows the effect of carnosine and acetylcarnosine on contact hypersensitivity to oxazolone.

### EXPERIMENTAL DETAILS

1. Methods

1.1 Solar simulated radiation (SSUV) was produced from a filtered fluorescent tube source, and was administered at the rate of 1 MED daily for 3Days.

1.2 Erythema was measured as a mid-dorsal skinfold thickness, which comprises mostly the oedema component of erythema.

1.3 Contact hypersensitivity was induced by application of 100 μl 0.2% w/v oxazolone in ethanol, to the abdominal skin of groups of 6 mice. The mice were challenged by application of 5 μl of fresh oxazolone/ethanol to each surface of both pinnae one week later, and the ear swelling response was measured 24 h later.

1.4 Collagen cross-linking by UV (solar) irradiation was achieved as described in WO 90/06102.

### 2. Results

2.1 Erythema

Carnosine incorporated into cosmetic creams at 0.1% and 1.00%, was compared with a cream containing 0.1% acetylcarnosine and a control cream containing neither carnosine nor acylcarnosine. The creams were applied daily (100 μl per mouse) immediately following irradiation with 1 MED of simulated solar UV radiation on 3 consecutive days only.

The sunburn (erythema) response was measured in mice by the oedema component at the peak of the erythema (48 h after the first UV exposure). There was significant reduction by the carnosine and acetylcarilosine creams, most effectively by 1% carnosine (see Table 1).

TABLE 1
Erythema/oedema as mid-dorsal skinfold thickness at 48 h

| | % increase in skinfold |
|---|---|
| Treatment | thickness* |
| Base + SSUV | 30.2 |
| 0.1% carnosine + SSUV | 6.0 |
| 1% carnosine + SSUV | 1.7 |
| 0.1% acetylcarnosine + SSUV | 11.3 |

*The percentage increase in skinfold thickness is a known standard method of measusring skin erythema/oedema

Quantitatively similar results were obtained in trials using skin on the backs of human volunteers.

2.2 Contact Hypersensitivity

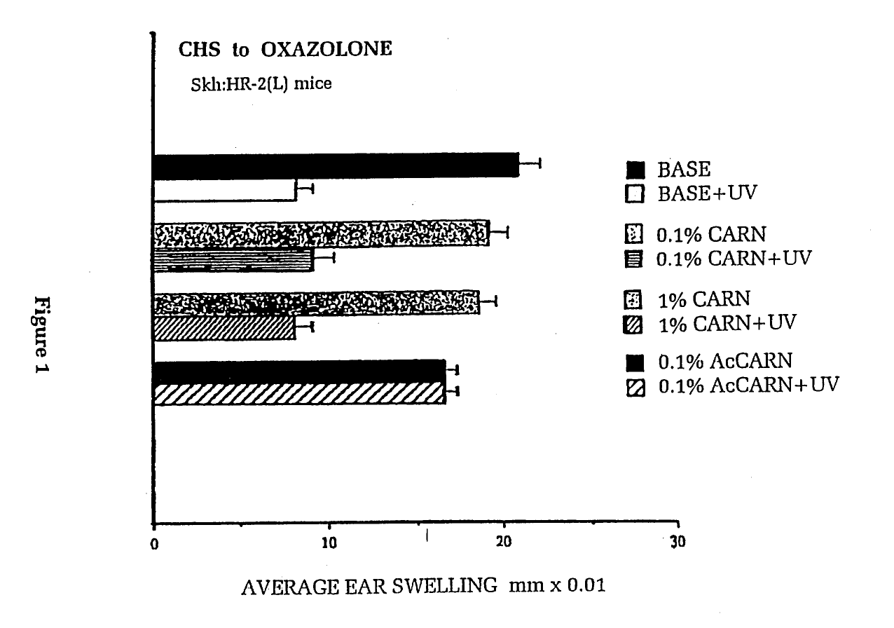

Contact hypersensitivity to oxazolone was induced after 1 week Marked suppression of the response (60.5%) was seen in mice irradiated through base (the cream vehicle), and there was no significant protection by the carnosine creams (51.6% and 55.9% respectively). However 0.1% acetylcarnosine totally prevented the suppression of contact hypersensitivity resulting from UV irradiation (see FIG. 1).

2.3 Collagen Cross Linking

Concentrations of 1-2 μM of acetyl carnosine completely inhibited cross linking of collagen by UV (solar) irradiation of collagen in human and mouse fibroblasts in vitro and in mouse skin.

2.4 Repair of Acute Sun Damage to Human Skin by Carnosine.

Four males aged 75, 74, 11 and 9 years were used in these studies. The 75 and 74 year olds were of fair (Celtic) complexion whereas the boys while not having an olive complexion did tan.

Case 1—Post-irradiaton treatment by a carnosine cream

Weather—full sunshine, time—December (mid summer). Place—Sydney.

The two boys spent a weekend on a yacht cruising in Sydney waters. No sun screen was used and boys wore only swimming briefs. After 2.5 full days of exposure their skin was quite red and tender to touch. They were treated with two applications of an (oil in water) emulsion containing 1% carnosine 3 hours apart before retiring to sleep on the third night. Next morning the erythema and skin tenderness had disappeared and the skin appeared normal. There was no subsequent speckling of the skin.

Case 2—Pre- and post-irradiation treatment by a carnosine cream

Weather—full sunshine, time—early May, place—Sydney.

Male aged 74 sailing on a yacht from 10 am to 4.30 pm. Skin protected only by all oil in water emulsion containing 1% carnosine. Very little erythema was apparent on his return home. A further treatment of the carnosine cream was applied before returning for the night to sleep. The next morning the skin was quite normal in appearance. This subject has a skin which is very sensitive to exposure to solar radiation and without taking particular precautions, invariably became badly sun burnt if he spent the day on the water sailing.

Case 3—Post-irradiation treatment by a carnosine cream

Weather—full sunshine, time—December 20th, place—Sydney

Male aged 75 sitting on the deck of a motor cruiser mooring around Sydney Harbour from 10.30 am to 5.00 pm. Subject had a sun-sensitive skin which became sun burnt after being exposed to water-reflected direct-solar radiation. Carnosine (1%) lotion was applied twice the next day to the effected areas on face and arms. By evening, the erythema had completely disappeared and no other effects were experienced.

Summary: In all cases the carnosine emulsion was effective in reducing or preventing all indications of erythema produced by solar radiation.

The results presented herein show that carnosine and its acylderivatives are surprisingly effective in the treatment of UV-induced erythema whether administered before and/or after exposure to UV radiation. These results suggest that carnosine and its acyl derivatives will be useful as sunscreen agents and in therapeutic compositions for treating damage caused by exposure to UV radiation. It is interesting to note that carnosine and its acyl derivatives do not absorb electromagnetic radiation in the UV-A or UV-B wave bands. Accordingly, unlike other known sunscreen agents, they do not have the disadvantage of potentiating the production of oxidative radicals in cells and tissues which may have absorbed these compounds.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Parrish et al. (1993) Photoimmunology, eds. Parrish, Kripke, Morison, Plenum Publishing Corporation, New York

2. Kripke, M. L. (1984) Immunol. Rev. 80:87-102

3. Schwartz et al. (1986) J. Invest. Dermatol. 87:289-291

4. Kim, T-Y. et al. (1990) J. Invest. Dermatol. 94:26-32

5. Ullrich, S. E. et al. (1990) J. Immunuol. 145:489-498

6. Reeve et al.(1993) Immunology 78:99-104

1. A method for preventing erythema or inflammatory cascade effects caused by solar radiation in a subject which method comprises administering to the subject at least one compound selected from the group consisting of carnosine, a compound related to carnosine, an acylcarnosine and a compound related to an acylcarnosine.

2. A method as claimed in claim 1 wherein the compound is administered after the subject has been exposed to solar radiation.

3. A method as claimed in claim 1 or claim 2 wherein the compound related to carnosine is selected from the group consisting of anserine, ophidine, homocarnosine, homoanserine, D-carnosine and carcinine.

4. A method for the treatment or prevention of UV induced suppression of an immune response in a subject, which method comprises administering to the subject a therapeutically or prophylactically effective amount of at least one acylcarnosine or a compound related to an acylcarnosine.

5. A method for the treatment or prevention of UV induced suppression of a T-cell mediated immune response in a subject, which method comprises administering to the subject a therapeutically or prophylactically effective amount of at least one acylcarnosine or a compound related to an acylcarnosine.

6. A method for preventing sunburn effects of UV radiation in a subject which method comprises administering to the subject at least one acylcarnosine or a compound related to an acylcarnosine.

7. A method for reducing cross-linking of collagen molecules in skin and/or damage to skin cell DNA which method comprises administering to the skin at least one acylcarnosine or a compound related to an acylcarnosine.

8. A method as claimed in claim 7 wherein the cross-linking is induced by UV radiation.

9. A method as claimed in any one of claims 4 to 8 wherein the acylacarnosine is selected from the group consisting of N-acetylcarnosine, N-butyl-carnosine, propionyl-carnosine and hexyl-carnosine.

10. A method as claimed in any one of claims 4 to 8 wherein the compound related to an acylacarnosine is an esters of an acylcarnosine.

11. A method as claimed in any one of claims 1 to 10 which further comprises administering topically to the subject a sunscreen agent.
